(19) United States
(12) Patent Application Publication
(10) Pub. No.: US 2007/0265526 A1
(43) Pub. Date: Nov. 15, 2007

(54) LOW-PROFILE LOCATION PAD

(76) Inventors: Assaf Govari, Haifa (IL); Andres Claudio Altmann, Haifa (IL); Yaron Ephrath, Kakur (IL)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/382,830

(22) Filed: May 11, 2006

(51) Int. Cl. A61B 5/05

(52) U.S. Cl. 600/424

(57) ABSTRACT

A magnetic position tracking system for performing a medical procedure on a patient who is positioned on an upper surface of a table includes a location pad, which is positioned on the upper surface of the table beneath the patient. The location pad includes one or more field generators, which are operative to generate respective magnetic fields and are arranged so that a thickness dimension of the location pad is no greater than 3 centimeters. A position sensor is fixed to an invasive medical device for insertion into a body of the patient, and is arranged to sense the magnetic fields so as to measure a position of the medical device in the body.

### FIELD OF THE INVENTION

The present invention relates generally to position tracking systems, and particularly to location pads used in magnetic position tracking.

### BACKGROUND OF THE INVENTION

Various methods and systems are known in the art for tracking the coordinates of objects involved in medical procedures. Some of these systems use magnetic field measurements. For example, U.S. Pat. Nos. 5,391,199 and 5,443,489, whose disclosures are incorporated herein by reference, describe systems in which the coordinates of an intrabody probe are determined using one or more field transducers. Such systems are used for generating location information regarding a medical probe, such as a catheter. A position sensor, such as a coil, is placed in the probe and generates signals in response to externally-applied magnetic fields. The magnetic fields are generated by magnetic field transducers, such as radiator coils, fixed to an external reference frame in known, mutually-spaced locations. The CARTO system, for example, produced by Biosense Webster Inc. (Diamond Bar, Calif.), operates in this manner.

Additional methods and systems that relate to magnetic position tracking are also described, for example, in PCT Patent Publication WO 96/05768, U.S. Pat. Nos. 6,690,963, 6,484,118, 6,239,724, 6,618,612 and 6,332,089, and U.S. Patent Application Publications 2002/0065455 A1, 2003/0120150 A1 and 2004/0068178 A1, whose disclosures are all incorporated herein by reference. These publications describe methods and systems that track the position of intrabody objects such as cardiac catheters, orthopedic implants and medical tools used in different medical procedures.

In some of the references cited above, the field transducers that generate the magnetic fields comprise field generating coils arranged in a coplanar, triangular arrangement. Another planar arrangement is described in U.S. Pat. No. 6,615,155, whose disclosure is incorporated herein by reference. The patent describes a system for tracking a moving object having a single field sensor using a set of non-overlapping planar loop antennas.

### SUMMARY OF THE INVENTION

Some embodiments of the present invention provide location pads comprising multiple magnetic field generators having a low profile (i.e., a reduced height dimension). This arrangement is advantageous in a number of medical position tracking applications. For example, a thin horizontal array of field generators can be conveniently placed on an operating or catheterization table, directly underneath a region of interest of a patient's body. A position sensor in a medical probe, such as a catheter, or other object in the body senses the fields produced by the field generators and outputs signals that are processed to determine coordinates of the probe or other object.

In some embodiments, the location pad includes two or more field generators arranged in a common plane. The field generators are oriented so as to generate respective magnetic fields perpendicular to the plane. In alternative embodiments, the field generators are oriented at known angles with respect to one another, without exceeding the overall low profile dimensions of the pad.

In some cases, however, the proximity of the field generators to the tracked position sensor may degrade the position measurement accuracy. For example, when the field generators comprise coils wound in the horizontal plane in order to reduce their height, measurements of vertical displacements of position sensors adjacent to this plane can be inaccurate.

To overcome this difficulty, in some embodiments of the present invention, the location pad comprises an auxiliary field generator, either within or outside of the location pad, which is oriented so as to generate an auxiliary magnetic field having a component parallel to the plane of the location pad at locations within the plane. By having the position sensor sense the two or more magnetic fields as well as the auxiliary magnetic field, the position tracking system is able to accurately measure the position of the position sensor even when it is located adjacent to the plane of the location pad.

In some embodiments, the position tracking system assists a physician in performing cardiac catheterization procedures. In these embodiments, a low profile location pad is placed on top of the catheterization table, underneath the patient's torso. A position sensor is fitted in the catheter inserted into the patient's heart. An additional reference position sensor may be attached to the patient's body, typically on the patient's back. Both sensors, and in particular the reference sensor, are adjacent to the plane of the location pad. Using the methods and systems described herein, the position tracking system can accurately track the positions of the catheter and the reference sensor.

In alternative embodiments of the present invention, a miniature magnetic field generator is attached to the catheter (or other object in the body), and the coils or other antennas in the location pad are used to sense the magnetic field and thus to determine the coordinates of the catheter (or other object). The novel principles of the design of the location pad are equally applicable in these embodiments in which the location pad serves as a receiver as in the embodiments in which it generates the magnetic fields.

There is therefore provided, in accordance with an embodiment of the present invention, a magnetic position tracking system for use in performing a medical procedure on a patient who is positioned on an upper surface of a table, the system including:

a location pad, which is positioned on the upper surface of the table beneath the patient, and which includes one or more field generators, which are operative to generate respective magnetic fields and are arranged so that a thickness dimension of the location pad is no greater than 3 centimeters; and

a position sensor, which is fixed to an invasive medical device for insertion into a body of the patient, and which is arranged to sense the magnetic fields so as to measure a position of the medical device in the body.

In an embodiment, the one or more field generators include two or more field generators positioned in a common plane and oriented so that the respective magnetic fields are perpendicular to the plane, and the system includes an auxiliary field generator oriented so as to generate an auxiliary magnetic field having a component parallel to the plane at locations within the common plane. In another embodiment, the location pad contains the auxiliary field generator without exceeding the thickness dimension.

In yet another embodiment, the two or more field generators include field generating coils wound in parallel to the common plane, and the auxiliary field generator includes a field generating coil wound not in parallel to the common plane.

In still another embodiment, the system includes a reference position sensor, which is attached to the body of the patient at a reference location adjacent to the common plane and is arranged to sense the auxiliary magnetic field and the magnetic fields generated by the two or more field generators so as to measure a position of the reference location. The reference position sensor may be arranged to accurately measure a vertical displacement of the reference location based on the sensed auxiliary magnetic field.

In an embodiment, the system includes a processor, which is arranged to calculate a position of the invasive medical device with respect to the body irrespective of patient movements based on magnetic field measurements provided by the position sensor fixed to the invasive medical device and by the reference position sensor.

In another embodiment, the one or more field generators include two or more field generators positioned at known angles with respect to one another.

There is also provided, in accordance with an embodiment of the present invention a magnetic position tracking system for use in performing a medical procedure on a patient who is positioned on an upper surface of a table, the system including:

a field generator, which is fixed to an invasive medical device for insertion into a body of the patient, and which is arranged to generate a magnetic field; and

a location pad, which is positioned on the upper surface of the table beneath the patient, and which includes one or more position sensors, which are operative to sense the magnetic field so as to measure a position of the medical device in the body, and are arranged so that a thickness dimension of the location pad is no greater than 3 centimeters.

There is further provided, in accordance with an embodiment of the present invention, a location pad for use in a magnetic position tracking system, including:

one or more primary field generators, which are arranged in a common plane and are oriented so as to generate respective primary magnetic fields perpendicular to the plane; and

an auxiliary field generator oriented to generate an auxiliary magnetic field having a component at locations within the plane that is parallel to the plane,

wherein the primary and auxiliary field generators are arranged so that a thickness dimension of the location pad is no greater than 3 centimeters.

There is additionally provided, in accordance with an embodiment of the present invention, a method for performing a medical procedure on a patient who is positioned on an upper surface of a table, the method including:

positioning on the upper surface of the table beneath the patient a location pad including one or more field generators operative to generate respective magnetic fields and arranged so that a thickness dimension of the location pad is no greater than 3 centimeters; and

sensing the magnetic fields by a position sensor fixed to an invasive medical device inserted into a body of the patient, so as to measure a position of the medical device in the body.

There is also provided, in accordance with an embodiment of the present invention, a method for performing a medical procedure on a patient who is positioned on an upper surface of a table, the method including:

generating a magnetic field by a field generator fixed to an invasive medical device inserted into a body of the patient; and

positioning on the upper surface of the table beneath the patient a location pad including one or more position sensors, which are operative to sense the magnetic field so as to measure a position of the medical device in the body and are arranged so that a thickness dimension of the location pad is no greater than 3 centimeters.

There is additionally provided, in accordance with an embodiment of the present invention, a method for position tracking, including:

positioning and orienting one or more primary field generators in a common plane so as to generate respective primary magnetic fields perpendicular to the plane; and

positioning and orienting an auxiliary field generator to generate an auxiliary magnetic field having a component at locations within the plane that is parallel to the plane,

wherein the primary and auxiliary field generators are arranged so that a thickness dimension of the location pad is no greater than 3 centimeters.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a magnetic position tracking system, in accordance with an embodiment of the present invention;

FIGS. 2A and 2B are diagrams that schematically illustrate a low profile location pad, in accordance with an embodiment of the present invention; and

FIG. 3 is a flow chart that schematically illustrates a method for magnetic position tracking, in accordance with an embodiment of the present invention.

### DETAILED DESCRIPTION OF EMBODIMENTS

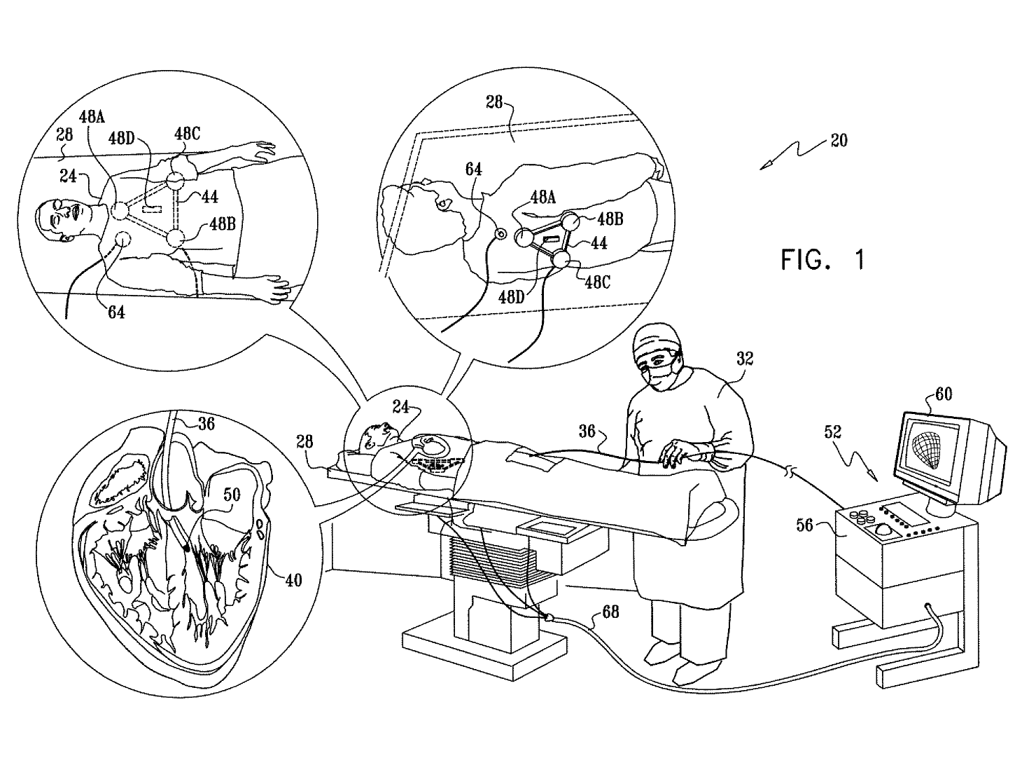

FIG. 1 is a schematic, pictorial illustration of a magnetic position tracking system 20 used in cardiac catheterization applications, in accordance with an embodiment of the present invention. A patient 24 lies on a catheterization table 28. A physician 32 inserts a catheter 36 into a chamber of a heart 40 of the patient. System 20 determines and displays the position and orientation coordinates of catheter 36 inside the heart.

System 20 comprises a location pad 44, which comprises one or more field generators, such as field generating coils. In the exemplary configuration of FIG. 1, pad 44 comprises four field generating coils 48A . . . 48D. The location pad is placed on top of the catheterization table under the patient's torso, such that coils 48A . . . 48D are located in fixed, known positions external to the patient. The field generating coils generate magnetic fields in a predefined working volume around heart 40.

The operation of system 20 is similar to that of the above-mentioned CARTO system, except that CARTO uses a bulky arrangement of field generator coils, which must be specially mounted beneath the catheterization table. Placing a thin location pad on the table obviates the need for special mounting and permits the pad to be moved freely from table to table. Placing the location pad on top of the table also increases the volume in which accurate position measurements can be performed, and may sometimes enable reducing the size of the position sensor. Additionally, when placed on top of the table, the location pad is typically further away from interfering objects, such as C-arm fluoroscopes, which may degrade the accuracy of position measurements.

A position sensor 50 fitted in the distal end of catheter 36 senses the magnetic fields in its vicinity. The position sensor produces and transmits, in response to the sensed fields, position signals to a console 52. The console comprises a tracking processor 56 that calculates the location and orientation of catheter 36 with respect to coils 48A . . . 48D, based on the position signals sent by position sensor 50. Typically, the console also drives coils 48A . . . 48D to generate the appropriate magnetic fields. The location and orientation coordinates of catheter 36 are displayed to the physician using a display 60.

In some embodiments, a reference position sensor 64 is attached to the patient's back and is used for calibration purposes. By tracking the position of reference sensor 64, system 20 can measure movements of the patient with respect to location pad 44 and can thus separate these movements from the movement of catheter 36 inside the patient's body. In other words, by measuring the relative position of sensor 50 with respect to reference sensor 64, system 20 can accurately determine the position of the catheter in the heart, regardless of patient movements, due to shifting of the patient's body or to respiration, for example. Sensor 64 and location pad 44 are connected to console 52 using suitable cables 68.

In order to enable location pad 44 to be conveniently placed between the patient and the catheterization table, the location pad is produced having a low profile. The thickness of the pad does not exceed 3 cm, and is typically smaller than 2 cm.

In some situations, however, the low profile of the location pad may degrade the measurement accuracy of the system. For example, in order to minimize the thickness of location pad 44, three of the field generating coils denoted 48A . . . 48C comprise low profile coils, wound in the horizontal plane in order to reduce their height dimension. As such, coils 48A . . . 48C generate magnetic fields perpendicular to the horizontal plane. This arrangement of fields may produce inaccurate position measurements of position sensors located adjacent to the horizontal plane containing the field generator coils. In particular, it may be difficult to accurately measure vertical displacements of such sensors. As can be seen in the figure, reference position sensor 64 is located only slightly above the horizontal plane containing field generating coils 48A . . . 48C (referred to as the plane of the location pad). Thus, sensor 64 may have poor sensitivity to vertical displacements when the measurements are based only on the fields generated by field generating coils 48A . . . 48C.

In order to improve the measurement accuracy of system 20, and in particular vertical displacement measurements of reference position sensor 64, field generating coil 48D, referred to as an auxiliary coil, is added to location pad 44. Coils 48A . . . 48C are referred to as primary field generators and the fields they generate are referred to as primary magnetic fields. Auxiliary coil 48D is wound in a plane perpendicular to the plane of the location pad, i.e., a plane that contains the vertical axis. As such, the auxiliary field generated by coil 48D is parallel to the plane of the location pad. Position signals produced by reference position sensor 64 due to the field generated by coil 48D will thus give an accurate indication of the vertical displacement of sensor 64, even though sensor 64 is adjacent to the plane of location pad 44. As will be shown below, coil 48D is wound so as to conform to the low profile of the location pad.

In alternative embodiments, the windings of coil 48D can be oriented at other angles with respect to the plane of the location pad, as long as the coil is not parallel with the plane. In other words, the field generated by coil 48D at locations within the plane of the location pad should have at least a component that is parallel to the plane. This component should be substantial enough to permit the vertical displacement of sensor 64 to be determined accurately to within the specified resolution limits of system 20. Further alternatively, the field generating coils can be tilted or otherwise oriented at known angles with respect to the horizontal plane, as long as they do not exceed the low profile dimensions of the pad.

In the example of FIG. 1, location pad 44 comprises a total of four field generating coils, out of which one coil comprises an auxiliary coil. In alternative embodiments, pad 44 may comprise any number of primary and auxiliary field generating coils arranged in any suitable configuration. Further alternatively, pad 44 may comprise a single field generating coil and catheter 36 comprises multiple position sensors positioned at known relative offsets and/or orientations, so as to enable tracking of the catheter location.

In some embodiments, auxiliary coil 48D may be located externally to location pad 44, such as next to the patient's feet. In such embodiments, there is no strict requirement as to the mechanical dimensions of the auxiliary coil. The auxiliary coil should still be positioned and oriented so that the field it generates has a component parallel to the plane of the location pad.

Although FIG. 1 shows a system for cardiac catheterization, low profile location pad 44 can be used in any other position tracking application, such as for tracking orthopedic implants and medical tools, as described in the background references cited above. In the example of FIG. 1 the location pad is placed horizontally and has a reduced height or vertical dimension. The methods and devices described herein can be used to reduce any desired dimension of the location pad, as appropriate for the particular application.

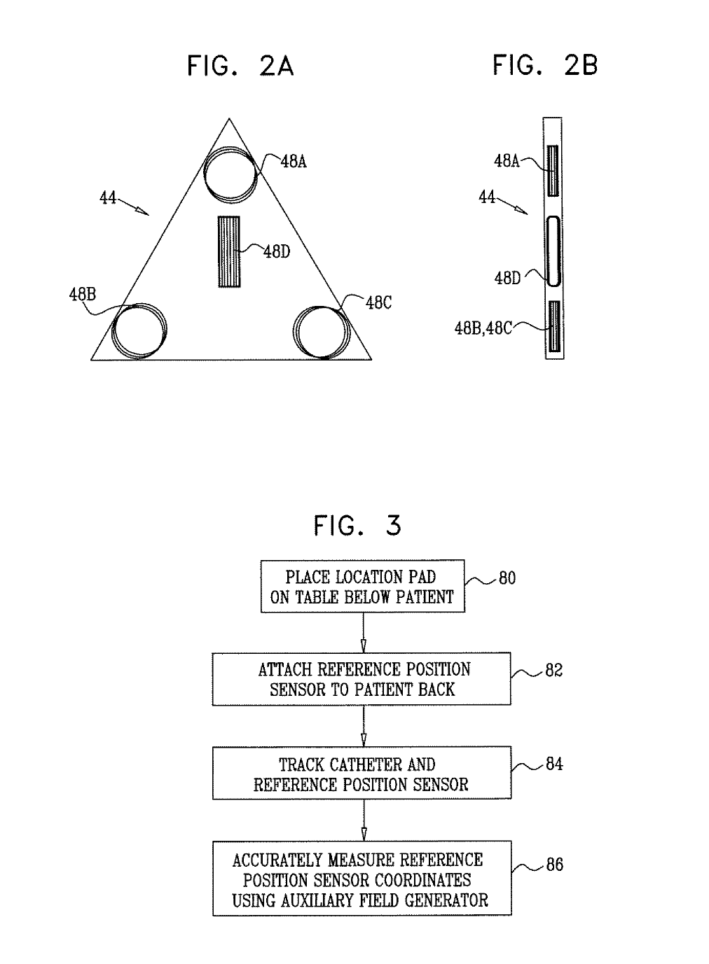

FIGS. 2A and 2B are diagrams that schematically illustrate low profile location pad 44, in accordance with an embodiment of the present invention.

FIG. 2A is a top view of pad 44, showing primary coils 48A . . . 48C arranged in a co-planar triangular configuration. The coils typically comprise air-wound coils, wound without a solid core or bobbin, so as to minimize their thickness. Alternatively, coils having cores that do not significantly increase the thickness of the pad can also be used. The distance between the coils is typically in the range of several centimeters to several tens of centimeters, although other distances can also be used. In the present example, the axis of auxiliary coil 48D is parallel to the plane of the location pad. Coil 48D is shown in the middle of the location pad, although it may alternatively be mounted at any other convenient location.

FIG. 2B is a side view of pad 44, showing coils 48A . . . 48D conforming to the overall low profile of the pad. In particular, coil 48D is shown to be wound using long and narrow windings, so as not to protrude beyond the thickness of primary coils 48A . . . 48C. The four coils may be fixed in their respective positions using a baseplate, frame or any other suitable fixture.

FIG. 4 is a flow chart that schematically illustrates a method for magnetic position tracking during a catheterization procedure, in accordance with an embodiment of the present invention. The method begins by placing location pad 44 on the catheterization table underneath the patient, at a pad positioning step 80. The physician attaches reference position sensor 64 to the patient's back, at a reference positioning step 82.

During the catheterization procedure, system 20 tracks position sensor 50 in catheter 36, as well as reference position sensor 64, at a tracking step 84. The four field generating coils 48A . . . 48D generate respective magnetic fields, which are sensed by position sensors 50 and 64. Tracking processor 56 accepts the position signals produced by sensors 50 and 64 responsively to the fields and calculates the position (location and orientation) coordinates of the two sensors. In particular, processor 56 accurately tracks the position of reference position sensor 64, at a reference tracking step 86. Using the field generated by auxiliary coil 48D, processor 56 is able to accurately determine the position of sensor 64, even though it is adjacent to the plane of the location pad.

Although the embodiments described hereinabove relate to a system in which a magnetic field is generated outside the patient's body and sensed by a position sensor in the body, in alternative embodiments the position sensor may be replaced by a miniature field generator in catheter 36, and coils 48A . . . 48D may be used instead to sense the field generated by the field generator in the catheter (and possibly by a reference field generator in place of sensor 64). Furthermore, although these embodiments refer mainly to a low profile location pad to be used in a magnetic position tracking system, the principles of the present invention can also be used in additional applications, such as in wireless power transfer applications in which electrical power is transferred to a low profile antenna pad.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art.

1. A magnetic position tracking system for use in performing a medical procedure on a patient who is positioned on an upper surface of a table, the system comprising:
a location pad, which is positioned on the upper surface of the table beneath the patient, and which comprises one or more field generators, which are operative to generate respective magnetic fields and are arranged so that a thickness dimension of the location pad is no greater than 3 centimeters; and
a position sensor, which is fixed to an invasive medical device for insertion into a body of the patient, and which is arranged to sense the magnetic fields so as to measure a position of the medical device in the body.

2. The system according to claim 1, wherein the one or more field generators comprise two or more field generators positioned in a common plane and oriented so that the respective magnetic fields are perpendicular to the plane, and comprising an auxiliary field generator oriented so as to generate an auxiliary magnetic field having a component parallel to the plane at locations within the common plane.

3. The system according to claim 2, wherein the location pad contains the auxiliary field generator without exceeding the thickness dimension.

4. The system according to claim 2, wherein the two or more field generators comprise field generating coils wound in parallel to the common plane, and wherein the auxiliary field generator comprises a field generating coil wound not in parallel to the common plane.

5. The system according to claim 2, and comprising a reference position sensor, which is attached to the body of the patient at a reference location adjacent to the common plane and is arranged to sense the auxiliary magnetic field and the magnetic fields generated by the two or more field generators so as to measure a position of the reference location.

6. The system according to claim 5, wherein the reference position sensor is arranged to accurately measure a vertical displacement of the reference location based on the sensed auxiliary magnetic field.

7. The system according to claim 5, and comprising a processor, which is arranged to calculate a position of the invasive medical device with respect to the body irrespective of patient movements based on magnetic field measurements provided by the position sensor fixed to the invasive medical device and by the reference position sensor.

8. The system according to claim 1, wherein the one or more field generators comprise two or more field generators positioned at known angles with respect to one another.

9. A magnetic position tracking system for use in performing a medical procedure on a patient who is positioned on an upper surface of a table, the system comprising:
a field generator, which is fixed to an invasive medical device for insertion into a body of the patient, and which is arranged to generate a magnetic field; and
a location pad, which is positioned on the upper surface of the table beneath the patient, and which comprises one or more position sensors, which are operative to sense the magnetic field so as to measure a position of the medical device in the body, and are arranged so that a thickness dimension of the location pad is no greater than 3 centimeters.

10. The system according to claim 9, wherein the one or more position sensors comprise two or more position sensors positioned in a common plane and oriented so as to sense components of the magnetic field perpendicular to the plane, and comprising an auxiliary position sensor oriented so as to sense a magnetic field component parallel to the plane.

11. A location pad for use in a magnetic position tracking system, comprising:
one or more primary field generators, which are arranged in a common plane and are oriented so as to generate respective primary magnetic fields perpendicular to the plane; and
an auxiliary field generator oriented to generate an auxiliary magnetic field having a component at locations within the plane that is parallel to the plane,
wherein the primary and auxiliary field generators are arranged so that a thickness dimension of the location pad is no greater than 3 centimeters.

12. A method for performing a medical procedure on a patient who is positioned on an upper surface of a table, the method comprising:
positioning on the upper surface of the table beneath the patient a location pad comprising one or more field generators operative to generate respective magnetic fields and arranged so that a thickness dimension of the location pad is no greater than 3 centimeters; and
sensing the magnetic fields by a position sensor fixed to an invasive medical device inserted into a body of the patient, so as to measure a position of the medical device in the body.

13. The method according to claim 12, wherein the one or more field generators comprise two or more field generators, and wherein positioning the location pad comprises positioning the two or more field generators in a common plane and orienting the two or more field generators so that the respective magnetic fields are perpendicular to the plane, and comprising positioning and orienting an auxiliary field generator so as to generate an auxiliary magnetic field having a component parallel to the plane at locations within the common plane.

14. The method according to claim 13, wherein positioning the location pad comprises installing the auxiliary field generator in the location pad without exceeding the thickness dimension.

15. The method according to claim 13, wherein the two or more field generators and the auxiliary field generator comprise field generating coils having windings, and wherein positioning the location pad comprises orienting the windings of the two or more field generators in parallel to the common plane and orienting the windings of the auxiliary field generator in an angle not in parallel to the common plane.

16. The method according to claim 13, and comprising attaching a reference position sensor to the body of the patient at a reference location adjacent to the common plane, and sensing the auxiliary magnetic field and the magnetic fields generated by the two or more field generators by the reference position sensor so as to measure a position of the reference location.

17. The method according to claim 16, wherein sensing the auxiliary magnetic field comprises accurately measuring a vertical displacement of the reference location based on the sensed auxiliary magnetic field.

18. The method according to claim 16, and comprising calculating a position of the invasive medical device with respect to the body irrespective of patient movements based on magnetic field measurements provided by the position sensor fixed to the invasive medical device and by the reference position sensor.

19. The method according to claim 12, wherein the one or more field generators comprise two or more field generators positioned at known angles with respect to one another.

20. A method for performing a medical procedure on a patient who is positioned on an upper surface of a table, the method comprising:
generating a magnetic field by a field generator fixed to an invasive medical device inserted into a body of the patient; and
positioning on the upper surface of the table beneath the patient a location pad comprising one or more position sensors, which are operative to sense the magnetic field so as to measure a position of the medical device in the body and are arranged so that a thickness dimension of the location pad is no greater than 3 centimeters.

21. The method according to claim 19, wherein the one or more position sensors comprise two or more position sensors, and wherein positioning the location pad comprises positioning the two or more position sensors in a common plane and orienting the two or more position sensors so as to sense components of the magnetic field perpendicular to the plane, and positioning and orienting an auxiliary position sensor so as to sense a component of the magnetic field parallel to the plane.

22. A method for position tracking, comprising:
positioning and orienting one or more primary field generators in a common plane so as to generate respective primary magnetic fields perpendicular to the plane; and
positioning and orienting an auxiliary field generator to generate an auxiliary magnetic field having a component at locations within the plane that is parallel to the plane,
wherein the primary and auxiliary field generators are arranged so that a thickness dimension of the location pad is no greater than 3 centimeters.
